(19) United States
(12) Patent Application Publication
(10) Pub. No.: US 2007/0122473 A1
(43) Pub. Date: May 31, 2007

(54) Aminosugar and/or glycosaminoglycan composition having therapeutic use

(76) Inventors: Linda A. Felton, Albuquerque, NM (US); Jamie G. Barnhill, Albuquerque, NM (US)

Correspondence Address: EDWARD J. TIMMER, P. O. BOX 770, RICHLAND, MI, 49083, US

(21) Appl. No.: 11/595,821

(22) Filed: Nov. 10, 2006

Related U.S. Application Data
(60) Provisional application No. 60/735,764, filed on Nov. 12, 2005.

(51) Int. Cl. A61K 31/737; A61K 31/7008; A61K 9/20

(52) U.S. Cl. 424/464; 514/54; 514/62

(57) ABSTRACT

An embodiment of the invention provides a composition having one or more active ingredients such as an aminosugar, more particularly, glucosamine, glucosamine salt, and mixtures thereof, and a glycosaminoglycan, more particularly, chondroitin, chondroitin salts, and mixtures thereof having a therapeutic use and is stable for at least 24 months. Additionally, the composition includes a diluent, glidant, and lubricant. The invention also provides a method of treating a connective tissue disease, injury, or condition comprising administering to a mammal any one of the compositions described herein. The composition can be in tablet or capsule form.

### RELATED APPLICATION

This application claims benefits and priority of provisional application Ser. No. 60/735,764 filed Nov. 12, 2005.

### BACKGROUND OF THE INVENTION

1. Field of the Invention

The present invention relates to compositions useful in the treatment of a connective tissue disease, injury, or condition in mammals, and, in particular, to compositions useful in the treatment of osteoarthritis.

2. Description of Related Art

The connective tissues such as cartilage, fibrocartilage, synovial membrane, ligament and the like are often subjected to mechanical stresses and forces, which can lead to injury and inflammation. Over time repeated injury can cause pain, swelling, stiffness and often results in arthritis or other degenerative conditions. These pathologies are very frequent and affect all types of mammals.

Treatment of the connective tissue can be problematic in that resting of the connective tissue is often unrealistic. Therefore, anti-inflammatory compounds such as glucocorticosteroids and NSAIDs (nonsteroidal anti-inflammatory drugs such as salycilates, indoles, and related compounds, propionics, pyrazols, anthranylines and the like) are used to treat the symptoms of the condition, not the cause.

The anti-inflammatory compounds can relieve pain and reduce the inflammatory state of the connective tissue but do not repair the connective tissue and may actually lead to further deterioration of the connective tissue.

In response to exercise, stress, and injury, connective tissue can naturally produce large quantities of collagen and proteoglycans (PGs), which are the major components of connective tissue, for self repair. Collagen is manufactured from amino acids, in particular from proline, glycine and lysine, and its biosynthesis is stimulated by the presence of glucosamine. PGs are large macromolecular complexes that form the framework for the collagen formation and also hold water for flexibility, resiliency, and resistance to compression. PGs consist of modified long chains of sugars called glycosaminoglycans (GAG) such as hyaluronic acid, chondroitin sulfates or alternatively heparin, and of which glucosamine is a precursor. During the repair process, collagen and PGs form a matrix, which confers on the tissues their respective mechanical properties.

After repeated injuries and stresses, the demand for the precursors of both the collagen and PG compounds increases while the availability of the precursors can decrease. In addition, the process for the biosynthesis of collagen and PGs from the precursors is relatively long, which constitutes another hindrance to the self repair of the tissues.

An object of the invention is to provide a stable, therapeutic composition that can bring exogenous precursor molecules such as glucosamine and chondroitin into the body, where the composition is administered orally and that can be manufactured in capsule or tablet form.

### SUMMARY OF THE INVENTION

An embodiment of the invention provides a stable, therapeutic composition having one or more active ingredients such as an aminosugar, more particularly, glucosamine, glucosamine salt, and mixtures thereof, and/or a glycosaminoglycan, more particularly, chondroitin, chondroitin salts, and mixtures thereof. Additionally, the composition includes a diluent, glidant, and lubricant. Proportions of the active ingredients, diluent, glidant, and lubricant are controlled to provide stability, potency, content uniformity, and dissolution at the time of manufacture and for at least 24 months after the date of manufacture.

In an embodiment of the invention the composition can comprise, in weight percent of the composition, an aminosugar from about 10 to about 70 percent, a glycosaminoglycan from about 10 to about 70 percent, a diluent from about 5 to about 80 percent, a glidant from about 0.02 to about 2 percent, and a lubricant from about 0.25 to about 5 percent.

In another embodiment the composition can comprise, in weight percent of the composition, an aminosugar from about 20 to about 95 percent, a diluent from about 5 to about 95 percent, a glidant from about 0.02 to about 2 percent, and a lubricant from about 0.25 to about 5 percent.

In still another embodiment the composition can comprise, in weight percent of the composition, a glycosaminoglycan from about 20 to about 95 percent, a first diluent from about 5 to about 95 percent, a second diluent from about 5 to about 95 percent, a glidant from about 0.02 to about 2 percent, and a lubricant from about 0.25 to about 5 percent.

Another embodiment of the invention provides a method of treating a connective tissue disease, injury, or condition comprising administering to a mammal any one of the compositions described herein. The composition can be administered in tablet or capsule form.

### DESCRIPTION OF FIGURES

FIG. 1 shows the mean glucosamine hydrochloride percentage potency over 24 months in 16 lots of capsules, where at the time of manufacture each capsule theoretically comprises in weight percent glucosamine hydrochloride (49.5%), sodium chondroitin sulfate (43.2%), Emcocel 50 (6.3%), Cab-O-Sil M-5P (0.5%), and magnesium stearate (0.5%).

FIG. 2 shows the mean sodium chondroitin sulfate potency over 24 months in 16 lots of capsules, where at the time of manufacture each capsule theoretically comprises in weight percent glucosamine hydrochloride (49.5%), sodium chondroitin sulfate (43.2%), Emcocel 50 (6.3%), Cab-O-Sil M-5P (0.5%), and magnesium stearate (0.5%).

FIG. 3 shows the mean glucosamine hydrochloride percentage potency over 24 months in 30 lots of capsules, where at the time of manufacture each capsule theoretically comprises in weight percent glucosamine hydrochloride (54.9%), Avicel PH 302 (44.0%), Cab-O-Sil M-5P (0.55%), and magnesium stearate (0.55%).

FIG. 4 shows the mean sodium chondroitin sulfate capsules percentage potency over 24 months in 17 lots of capsules, where each capsule at the time of manufacture theoretically comprises in weight percent sodium chondroitin sulfate (43.2%), Emcompress (15.8%), Avicel PH 302 (40%), Cab-O-Sil M-5P (0.5%), and magnesium stearate (0.5%).

### DETAILED DESCRIPTION OF THE INVENTION

The invention provides a stable, therapeutic composition that is especially useful in the treatment of connective tissue disease, injury, or condition such as osteoarthritis but is not limited thereto as other diseases may benefit therefrom.

1. Aminosugar and Glycosaminoglycan Composition

An embodiment of the invention provides a composition comprising both an aminosugar and a glycosaminoglycan, both of which are active ingredients. The composition has a therapeutic use and comprises, in weight percent of the composition, an aminosugar from about 10 to about 70 percent, a glycosaminoglycan from about 10 to about 70 percent, a diluent from about 5 to about 80 percent, a glidant from about 0.02 to about 2 percent, and a lubricant from about 0.25 to about 5 percent. The composition is stable for at least 24 months after the date of manufacture.

The aminosugar can be selected from the group consisting of glucosamine, glucosamine salts, and mixtures thereof and is preferably glucosamine hydrochloride, which is commercially available from Ferro Pfanstiehl Laboratories, Inc. of Waukegon, Ill.

The glycosaminoglycan can be selected from a group consisting of chondroitin, chondroitin salts, and mixtures thereof, and is preferably sodium chondroitin sulfate, which is commercially available from Bioiberica of Barcelona, Spain.

The diluent can be selected from the group consisting of microcrystalline cellulose, starch, calcium phosphate, calcium carbonate, detroxe, detrines, dextrates, lactose, mannitol, sucrose, silicified microcrystalline cellulose, cellulose, and mixtures thereof. The diluent is preferably microcrystalline cellulose, which is commercially available as Emcocel 50 manufactured by JRS Pharma of Patterson, N.Y. or Avicel PH101 manufactured by FMC BioPolymer of Philadelphia, Pa.

The glidant can be selected from the group consisting of fumed silica, talc, cornstarch, magnesium, silicate, magnesium trisilicate, and mixtures thereof. The glidant is preferably fumed silica, which is commercially available as Cab-O-Sil M-5P manufactured by Cabot Corporation of Boston, Mass.

The lubricant can be selected from the group consisting of magnesium stearate, stearic acid, hydrogenated castor oil, talc, glycerin monostearate, magnesium lauryl sulfate, zinc stearate, polyethylene glycol, and mixtures thereof. The lubricant is preferably magnesium stearate (USP grade) commercially available from Spectrum Chemical and Laboratory Products, Inc of Gardena, Calif.

In a particularly preferred embodiment of the invention, the composition comprises, in weight percent of the composition, about 50 percent of an aminosugar, about 43 percent of a glycosaminoglycan, 6 percent of a diluent, about 0.5 percent of a glidant, about 0.5 percent of a lubricant.

An illustrative embodiment of the invention is a composition comprising, in weight percent of the composition, glucosamine hydrochloride in an amount of about 50 percent, sodium chondroitin sulfate in an amount of about 43 percent, microcrystalline cellulose in an amount of about 6 percent, fumed silica in an amount of about 0.5 percent, and magnesium stearate in an amount of about 0.5 percent.

The following example is offered in order to more fully illustrate this embodiment of invention but is not to be construed as limiting the scope thereof.

### EXAMPLE

The invention was practiced using batch sizes of 20 kg and 80 kg to demonstrate the manufacturability in the different batch sizes. The two sizes represent quantities needed for a clinical trial and the capacity of the available processing equipment. The batches comprised (percentages are in weight percent):

20 kg Batch Size

9.9 kg Glucosamine HCl (49.5%)

8.64 kg Sodium Chondroitin Sulfate (43.2%)

1.26 kg Emcocel 50 (6.3%)

0.1 kg Cab-O-Sil M-5P (0.5%)

0.1 kg Magnesium Stearate (0.5%)

80 kg Batch Size

39.6 kg Glucosamine HCl (49.5%)

34.56 kg Sodium Chondroitin Sulfate (43.2%)

5.04 kg Emcocel 50 (6.3%)

0.4 kg Cab-O-Sil M-5P (0.5%)

0.4 kg Magnesium Stearate (0.5%)

The sodium chondroitin sulfate was sieved through a 40 mesh screen and weighed to the appropriate amount. The glucosamine HCl was processed through a Fitzmill using a 40 mesh screen and weighed to the appropriate amount. The Emcocel 50 was seived through a 40 mesh screen and weighed to the appropriate amount. The Cab-O-Sil was weighed to the appropriate amount. The sodium chondroitin sulfate, glucosamine HCl, Emococel 50, and Cab-O-Sil were blended in a V-shell blender with an intensifier bar commercially available from Patterson Kelly of East Stroudsburg, Pa. or, for larger batches in a double ribbon blender commercially available from AIM Blending Technologies, Inc of Pleasanton, Calif. for 35 minutes to produce a homogenous blend. The magnesium stearate was sieved through a 40 mesh screen, weighed to the appropriate amount, and added to the other powders to form a mixture. The mixture was then blended for 5 minutes and then filled into hard gelatin capsules using an automatic encapsulation machine by Bosch to a powder fill weight of approximately 505 mg.

Samples of the powder blend were removed and assayed for potency and content uniformity prior to the addition of the magnesium stearate. If the results were not within acceptable limits, additional blending was necessary. It is critical to achieve a uniform blend before the addition of the lubricant since overblending of the lubricant could adversely affect dissolution.

The potency of the composition was determined by measuring the glucosamine and chondroitin concentrations in the capsule samples. The glucosamine concentration was measured by reverse phase high-pressure liquid chromatography (RV-HPLC) utilizing photodiode array detection at 195 nm. The mobile phase used in the glucosamine analysis consisted of a mixture of phosphate buffer (pH 3.0) and acetonitrile (3:2, V:V). The column was a Luna (Phenomenex) C18 250×4.6 mm 5 micron, equipped with a guard. The flow rate was 0.6 mL/min and the injection volume was 10 μL. Samples were prepared by combining the contents of 20 capsules. An amount of powder equivalent to 100 mg of glucosamine was transferred to a 100 mL volumetric. Samples were sonicated, centrifuged, filtered and analyzed. The acceptance criteria for glucosamine potency was set at 90.0-110.0% label amount.

The chondroitin concentration was measured by a titrimetric assay using cetylpyridinium chloride as the titrant. This method involved measuring the flocculation point using a phototrode. The potency assay was performed by dissolving one sample capsule in 900 ml water. A 50 ml aliquot was titrated with cetylpyridinium chloride after standardization of the titrant. A Brinkmann 726 Titroprocessor with phototrode was used for the analysis. The acceptance criteria for chondroitin potency was set at 85.0-115.0% label amount.

The content uniformity of the composition was determined following United States Pharmacopeia, hereinafter USP, guidelines as specified in section <905>.

Each capsule has a theoretical potency, which is the amount of each active ingredient (Glucosamine HCl and Sodium Chondroitin Sulfate) that is calculated to be in each capsule. For example, each capsule from the batches should theoretically contain (percentages are in weight percent):
250 mg Glucosamine HCl (49.5%);
218 mg Sodium Chondroitin Sulfate (43.2%, includes weight from water to provide 200 mg of chondroitin sulfate/cap);
32 mg Emcocel 50 (6.3%);
2.5 mg Cab-O-Sil M-5P (0.5%); and
2.5 mg Magnesium Stearate (0.5%).
   The percent of theoretical potency was then calculated by taking the average actual amount of the active ingredients divided by the theoretical potency (or label claim amount) then multiplied by 100 for percent. The percentage of theoretical potency can be over 100% because there is some variation in product within the batch (i.e. some capsules have more or less active ingredients than the theoretical potency).

The relative standard deviation between samples was calculated by dividing the standard deviation of the data by the average then multiplying by 100 and represents the content uniformity.

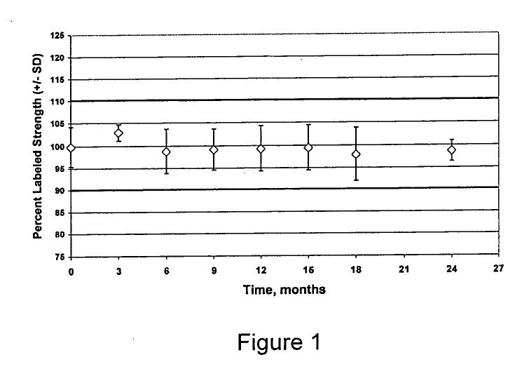

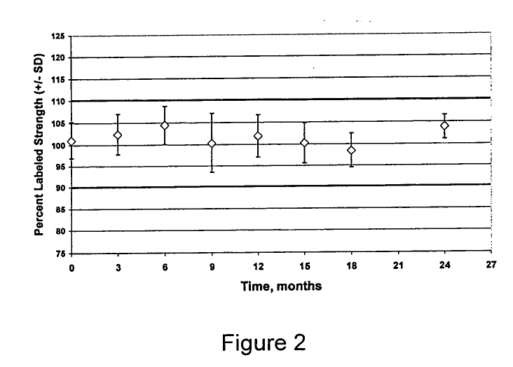

Samples of the final manufactured product were stored in sealed bottles in ambient conditions and tested for potency and dissolution over time. The potency of the active ingredients was measured as described above. A stable formulation is defined as one having 90% or more of theoretical potency for each of the active ingredients glucosamine and chondroitin sulfate. As shown in Tables 1, 2, and 3, the composition is stable for at least 24 months after the date of manufacture. FIGS. 1 and 2 are graphical representations of the data in table 3.

The dissolution of the active ingredients chondroitin sulfate and glucosamine tests the release of the active ingredient at a certain time and is important for quality assurance. The dissolution testing for all capsule products utilized USP Apparatus II with a 60 min run time. Both the glucosamine and the chondroitin used deionized water as media and samples were pooled. After pooling the dissolution samples, a 50 ml aliquot was titrated with cetylpyridinium chloride after standardization of the titrant, or was analyzed by HPLC analysis. The dissolution criteria was set at Q=75%, which means at least 75% of the drug should be released at the specified time (60 minutes), for both glucosamine and chondroitin. Tables 1 and 2 show dissolution after 60 minutes of the capsule sample being dissolved in deionized water. The percent dissolution can be over 100% since it is based on theoretical potency or label claim.

TABLE 1
The potency, content uniformity, and dissolution data for the 20 kg batch.

| | | | Chondroitin | Chondroitin |
|---|---|---|---|---|
| | Glucosamine | Chondroitin Sulfate | sulfate | sulfate |
| Timepoint | Potency | Potency | Content | Dissolution |
| (months) | (% of theoretical) | (% of theoretical | Uniformity | (at 60 min) |
| 0 | 271.0 mg (108.4%) | 207.1 mg (103.6%) | 1.0% RSD | 101.0% |
| 3 | 257.6 mg (103.1%) | 200.3 mg (100.2%) | | 104.2% |
| 6 | 248.1 mg (99.2%) | 214.7 mg (107.3%) | | 102.9% |
| 9 | 245.2 mg (98.1%) | 186.6 mg (93.3%) | | |
| 12 | 244.3 mg (97.7%) | 193.4 mg (96.7%) | | 94.5% |
| 18 | 264.8 mg (105.9%) | 198.2 mg (99.1%) | | 91% |

TABLE 2
The potency, content uniformity, and dissolutin data for the 80 kg batch.

| | | Chondroitin | | | |
|---|---|---|---|---|---|
| | Glucosamine | Sulfate | | | |
| Timepoint | Potency | Potency | | Content | Dissolution |
| months | (% of theoretical) | (% of theoretical) | | Uniformity | (at 60 min) |
| 0 | 252.7 mg (101.1%) | 210.1 mg | (105.0%) | 1.5% RSD glu* | 100.3% glu |
| | | | | 0.4% RSD ch* | 105.7% ch |
| 6 | 260.4 mg (104.2%) | 195.0 mg | (97.5%) | | 96.3%-ch |
| 9 | 238.3 mg (95.3%) | 190.7 mg | (95.3%) | | |
| 12 | 226.2 mg (90.5%) | 188.7 mg | (94.3%) | | 94.1%-ch |
| 18 | 272.8 mg (109.1%) | 193.1 mg | (96.5%) | | 91.8%-ch |
| 24 | 226.2 mg (90.5%) | 183.7% | (91.8%) | | 90.0%- ch |

*glu = glucosamine, ch = chondroitin

TABLE 3
The average percent potency and the relative standard
deviation of glucosamine and chondroitin sulfate of 16
lots taken from both the 20 kg and 80 kg batches.

| | | Standard | Chondroitin | Standard |
|---|---|---|---|---|
| Timepoint (months) | Glucosamine | Deviation | sulfate | Deviation |
| 0 | 99.8% | 4.4 | 101.0% | 4.2 |
| 3 | 102.9% | 1.8 | 102.4% | 4.7 |
| 6 | 98.7% | 5.0 | 104.4% | 4.3 |
| 9 | 99.1% | 4.6 | 100.3% | 6.9 |
| 12 | 99.2% | 5.0 | 101.9% | 4.9 |
| 18 | 97.9% | 5.9 | 98.5% | 4.0 |
| 24 | 98.6% | 2.3 | 103.8% | 2.8 |

2. Aminosugar Composition

Another embodiment of the invention provides a composition comprising an aminosugar as an active ingredient. The composition has a therapeutic use and comprises, in weight percent of the composition, an aminosugar from about 20 to about 95 percent, a diluent from about 5 to about 95 percent, a glidant from about 0.02 to about 2 percent, and a lubricant from about 0.25 to about 5 percent. The composition is stable for at least 24 months after the date of manufacture.

The aminosugar, diluent, glidant, and lubricant are described above. In a preferred embodiment the diluent is microcrystalline cellulose, commercially available as Avicel PH 302 manufactured by FMC BioPolymer; the glidant is fumed silica, commercially available as Cab-O-Sil M-5P manufactured by Cabot Corporation; and the lubricant is magnesium stearate (USP grade) commercially available from Spectrum Chemical and Laboratory Products, Inc.

In a particularly preferred embodiment of the invention, the composition comprises, in weight percent of the composition, 55 percent of an aminosugar, 44 percent of a diluent, 0.5 percent of a glidant, 0.5 percent of a lubricant. The aminosugar, diluent, glidant, and lubricant are described above.

An illustrative embodiment of the invention is a composition comprising, in weight percent of the composition, glucosamine hydrochloride in an amount of about 55 percent, microcrystalline cellulose in an amount of about 44 percent, fumed silica in an amount of about 0.5 percent, and magnesium stearate in an amount of about 0.5 percent.

The following example is offered in order to more fully illustrate this embodiment of the invention but is not to be construed as limiting the scope thereof.

### EXAMPLE

The invention was practiced using batch sizes of 20 kg and 80 kg to demonstrate the manufacturability of the different batch sizes. The two sizes represent quantities needed for a clinical trial and the capacity of the available processing equipment. The batches comprised (percentages are in weight percent):

20 kg Batch

10.98 kg Glucosamine HCl (54.9%)

8.8 kg Avicel PH 302 (44.0%)

0.11 kg Cab-O-Sil M-5P (0.55%)

0.11 kg Magnesium Stearate (0.55%)

80 kg Batch

43.92 kg Glucosamine HCl (54.9%)

35.2 kg Avicel PH 302 (44.0%)

0.44 kg Cab-O-Sil M-5P (0.55%)

0.44 kg Magnesium Stearate (0.55%)

The glucosamine HCl was processed through a Fitzmill using a 40 mesh screen and weighed to the appropriate amount. The Avicel PH 302 was seived through a 40 mesh screen and weighed to the appropriate amount. The Cab-O-Sil was weighed to the appropriate amount. The glucosamine HCl, Avicel PH 302, and Cab-O-Sil were blended in a V-shell blender with an intensifier bar commercially available from Patterson Kelly or, for larger batches a double ribbon blender commercially available from AIM for 25 minutes to produce a homogenous blend. The magnesium stearate was sieved through a 40 mesh screen, weighed to the appropriate amount, and added to the other powders to form a mixture. The mixture was then blended for 5 minutes and then filled into hard gelatin capsules using an automatic encapsulation machine by Bosch to a powder fill weight of approximately 455 mg.

Samples of the powder blend were removed and assayed for potency and content uniformity prior to the addition of the magnesium stearate. If the results were not within acceptable limits, additional blending was necessary. It is critical to achieve a uniform blend before the addition of the lubricant since overblending of the lubricant could adversely affect dissolution.

The potency of the composition was determined by measuring the glucosamine in the samples. The glucosamine concentration was measured by reverse phase high-pressure liquid chromatography (RV-HPLC) utilizing photodiode array detection at 195 nm. The mobile phase used in the glucosamine analysis consisted of a mixture of phosphate buffer (pH 3.0) and acetonitrile (3:2, V:V). The column was a Luna (Phenomenex) C18 250×4.6 mm 5 micron, equipped with a guard. The flow rate was 0.6 mL/min and the injection volume was 10 μL. Samples were prepared by combining the contents of 20 capsules. An amount of powder equivalent to 100 mg of glucosamine was transferred to a 100 mL volumetric. Samples were sonicated, centrifuged, filtered and analyzed. The acceptance criteria for glucosamine potency was set at 90.0-110.0% label amount.

The content uniformity of the samples was determined following United States Pharmacopeia, hereinafter USP, guidelines as specified in section <905>.

Each capsule has a theoretical potency, which is the amount of the active ingredient Glucosamine HCl calculated to be in each capsule. For example, each capsule from the batches should theoretically contain (percentages are in weight percent):

250 mg Glucosamine HCl (54.9%)

200 mg Avicel PH 302 (44.0%)

2.5 mg Cab-O-Sil M-5P (0.55%)

2.5 mg Magnesium Stearate (0.55%)

The percent of theoretical potency was then calculated by taking the average actual amount divided by the theoretical potency (or label claim amount) then multiplied by 100 for percent. The percentage of theoretical potency can be over 100% because there is some variation in product within the batch (i.e. some capsules have more or less active ingredients than the theoretical potency).

The relative standard deviation between samples was calculated by dividing the standard deviation of the data by the average then multiplying by 100.

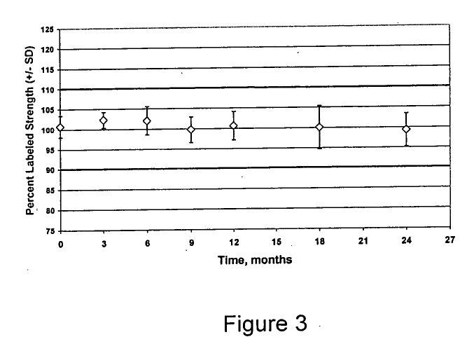

Samples of the final manufactured product were stored in sealed bottles in ambient conditions and tested for potency and dissolution over time. The potency of the glucosamine was measured as described above. A stable formulation is defined as one having 90% or more of theoretical potency for the active ingredient glucosamine. As shown in Tables 4-7, the composition is stable for at least 24 months after the date of manufacture. FIG. 3 is a graphical representation of the data in table 6.

The dissolution of the active ingredient glucosamine hydrochloride over time is shown in tables 4, 5, and 7 and tests the release of the active ingredient at a certain time and is important for quality assurance. The dissolution testing for all capsule products utilized USP Apparatus II with a 45 minute run time. The glucosamine dissolution used deionized water as media and samples were pooled. After pooling the dissolution samples, a 50 ml aliquot was titrated with cetylpyridinium chloride after standardization of the titrant, or was analyzed by HPLC analysis. The dissolution criteria was set at Q=75%, which means at least 75% of the glucosamine should be released at the specified time (45 minutes). The percent dissolution can be over 100% since it is based on theoretical potency or label claim.

TABLE 4
The potency, content uniformity, and
dissolution data for the 20 kg batch.

| | Glucosamine Potency | | Dissolution |
|---|---|---|---|
| Timepoint | (% of theoretical) | Content | (at 45 min) |
| (months) | Specification | uniformity | Q = 75% |
| 0 | 259.4 mg (103.8%) | 0.98% RSD | |
| 3 | 251.8 mg (100.7%) | | 108.6% |
| 6 | 256.0 mg (102.4%) | | 99.1% |
| 9 | 250.8 mg (100.3%) | | 125.4% |
| 12 | 227.1 mg (90.8%) | | 106.6% |
| 18 | 227.9 mg (91.1%) | | 95.8% |

TABLE 5
The potency, content uniformity, and
dissolution data for the 80 kg batch.

| | | | Dissolution |
|---|---|---|---|
| Timepoint | Glucosamine Potency | Content | (at 45 min) |
| (months) | (% of theoretical) | Unformity | Q = 75% |
| 0 | 245.3 mg (98.1%) | 1.13% RSD | |
| 6 | 243.2 mg (97.3%) | | 100.2% |
| 9 | 246.9 mg (98.8%) | | 94.0% |
| 12 | 249.0 mg (97.7%) | | 115.7% |
| 15 | 249.8 mg (99.9%) | | 106.6% |

TABLE 6
The average percent potency and relative standard
deviation of glucosamine hydrochloride of 30 lots
taken from both the 20 kg and 80 kg batches.

| Timepoint (months) | Average Potency | Standard Deviation |
|---|---|---|
| 0 | 100.7 | 2.7 |
| 3 | 102.3 | 2.0 |
| 6 | 102.1 | 3.5 |
| 9 | 99.7 | 3.2 |
| 12 | 100.7 | 3.6 |
| 18 | 100.0 | 5.4 |
| 24 | 99.3 | 4.1 |

TABLE 7
The average dissolution value of glucosamine hydrochloride
of 30 lots taken from both the 20 kg and 80 kg batches.

| | Average Dissolution | |
|---|---|---|
| Timepoint (months) | (Q = 75% at 45 min) | Standard Deviation |
| 0 | 95.2 | 10.0 |
| 3 | 104.9 | 2.2 |
| 6 | 103.1 | 3.1 |
| 9 | 105.4 | 4.4 |
| 12 | 102.7 | 3.3 |
| 18 | 99.6 | 6.6 |
| 24 | 102.7 | 4.7 |

3. Glycosaminoglycan Composition

Another embodiment of the invention provides a composition comprising a glycosaminoglycan as an active ingredient. The composition comprises, in weight percent of the composition, a glycosaminoglycan from about 20 to about 95 percent, a first diluent from about 5 to about 95 percent, a second diluent from about 5 to about 95 percent, a glidant from about 0.02 to about 2 percent, and a lubricant from about 0.25 to about 5 percent. The composition is stable for at least 24 months after the date of manufacture.

The glycosaminoglycan, glidant, and lubricant are described above.

The first diluent is preferably dibasic calcium phosphate but can also be starch, microcrystalline cellulose, calcium carbonate, dextroxe, dextrines, dextrates, lactose, mannitol, sucrose, silicified microcrystalline cellulose, cellulose, and mixtures thereof.

The second diluent is preferably microcrystalline cellulose but can also be starch, calcium phosphate, calcium carbonate, detroxe, detrines, dextrates, lactose, mannitol, sucrose, silicified microcrystalline cellulose, cellulose, and mixtures thereof.

In a preferred embodiment the first diluent is dibasic calcium phosphate, which is commercially available as Emcompress manufactured by JRS Pharma; the second diluent is microcrystalline cellulose, which is commercially available as Avicel PH 302 manufactured by FMC BioPolymer or as Emcocel from JRS Pharma; the glidant is fumed silica, which is commercially available as Cab-O-Sil M-5P manufactured by Cabot Corporation; and the lubricant is magnesium stearate (USP grade) commercially available from Spectrum Chemicals.

In a particularly preferred embodiment of the invention, the composition comprises, in weight percent of the composition, 43 percent of a glycosaminoglycan, 16 percent of a first diluent, 40 percent of a second diluent, 0.5 percent of a glidant, and 0.5 percent of a lubricant.

An illustrative embodiment of the invention is a composition comprising, in weight percent of the composition, sodium chondroitin sulfate in an amount of about 43 percent, dibasic calcium phosphate in an amount of about 16 percent, microcrystalline cellulose in an amount of about 40 percent, fumed silica in an amount of about 0.5 percent, and magnesium stearate in an amount of about 0.5 percent.

The following example is offered in order to more fully illustrate this embodiment of the invention but is not to be construed as limiting the scope thereof.

### EXAMPLE

The invention was practiced using two different batch sizes of 20 kg and 80 kg to demonstrate the manufacturability of the different batch sizes. The two sizes represent quantities needed for a clinical trial and the capacity of the available processing equipment. The batches comprised (percentages are in weight percent):

20 kg Batch
8.64 kg Sodium Chondroitin Sulfate (43.2%, includes weight from water to provide 200 mg of chondroitin sulfate/cap)
31.6 kg Emcompress (15.8%)
8 kg Avicel PH 302 (40%)
0.1 kg Cab-O-Sil M-5P (0.5%)
0.1 kg Magnesium Stearate (0.5%)

80 kg Batch
34.56 kg Sodium Chondroitin Sulfate (43.2%, includes weight from water to provide 200 mg of chondroitin sulfate/cap)
12.64 kg Emcompress (15.8%)
32 kg Avicel PH 302 (40%)
0.4 kg Cab-O-Sil M-5P (0.5%)
0.4 kg Magnesium Stearate (0.5%)

The sodium chondroitin sulfate was sieved through a 40 mesh screen and weighed to the appropriate amount. The Emcompress and Avicel PH 302 were each sieved through a 40 mesh screen and weighed to the appropriate amounts. The Cab-O-Sil was weighed to the appropriate amount. The sodium chondroitin sulfate, Emcompress, Avicel PH 302, and Cab-O-Sil were blended in a V-shell blender with an intensifier bar commercially available from Patterson Kelly or, for larger batches in a double ribbon blender commercially available from AIM.

The magnesium stearate was sieved through a 40 mesh screen, weighed to the appropriate amount, and added to the other powders to form a mixture. The mixture was then blended for 5 minutes and then filled into hard gelatin capsules using an automatic encapsulation machine by Bosch to a powder fill weight of approximately 505 mg.

Samples of the powder blend were removed and assayed for potency and content uniformity prior to the addition of the magnesium stearate. If the results were not within acceptable limits, additional blending was necessary. It is critical to achieve a uniform blend before the addition of the lubricant since overblending of the lubricant could adversely affect dissolution.

The potency of the composition was determined by measuring the chondroitin concentration in the samples. The chondroitin concentration was measured by a titrimetric assay using cetylpyridinium chloride as the titrant. This method involved measuring the flocculation point using a phototrode. The potency assay was performed by dissolving one sample capsule in 900 ml water. A 50 ml aliquot was titrated with cetylpyridinium chloride after standardization of the titrant. A Brinkmann 726 Titroprocessor with phototrode was used for the analysis. The acceptance criteria for chondroitin potency was set at 85.0-115.0% label amount.

The content uniformity of the composition was determined following United States Pharmacopeia, hereinafter USP, guidelines as specified in section <905>.

Each capsule has a theoretical potency, which is the amount of the active ingredient Chondroitin Sulfate calculated be in each capsule. For example, each capsule from the batches should theoretically contain (percentages are in weight percent):
218 mg Sodium Chondroitin Sulfate (43.2%, includes weight from water to provide 200 mg of chondroitin sulfate/cap)
80 mg Emcompress (15.8%)
202 mg Avicel PH 302 (40%)
2.5 mg Cab-O-Sil M-5P (0.5%)
2.5 mg Magnesium Stearate (0.5%)

The percent of theoretical potency was then calculated by taking the average actual amount divided by the theoretical potency (or label claim amount) then multiplied by 100 for percent. The percentage of theoretical potency can be over 100% because there is some variation in product within the batch (i.e. some capsules have more or less active ingredients than the theoretical potency).

The relative standard deviation between samples was calculated by dividing the standard deviation of the data by the average then multiplying by 100.

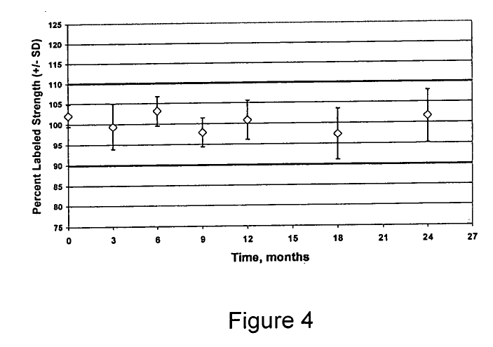

Samples of the final manufactured product were stored in sealed bottles in ambient conditions and tested for potency and dissolution over time. The potency of the chondroitin was measured as described above. A stable formulation is defined as one having 90% or more of theoretical potency for the active ingredient chondroitin sulfate. As shown in Tables 8-11, the composition is stable for at least 24 months after the date of manufacture. FIG. 4 is a graphical representation of the data in table 10.

The dissolution of the active ingredients chondroitin sulfate and glucosamine tests the release of the active ingredient at a certain time and is important for quality assurance. The dissolution testing for all capsule products utilized USP Apparatus II with a 60 min run time. Both the glucosamine and the chondroitin used deionized water as media and samples were pooled. After pooling the dissolution samples, a 50 ml aliquot was titrated with cetylpyridinium chloride after standardization of the titrant, or was analyzed by HPLC analysis. The dissolution criteria was set at Q=75%, which means at least 75% of the drug should be released at the specified time (60 minutes), for both glucosamine and chondroitin. The dissolution of the active ingredient chondroitin sulfate over time is shown in tables 8, 9, and 11. The percent dissolution can be over 100% since it is based on theoretical potency or label claim.

TABLE 8
The potency, content uniformity, and
dissolution data for the 20 kg batch.

| | Chondroitin Sulfate | | Dissolution |
|---|---|---|---|
| Timepoint | Potency | Content | Q = 75% |
| (months) | (% of theoretical) | Uniformity | (at 60 min) |
| 0 | 200.2 mg (99.9%) | 1.65% RSD | |
| 1 | 201.9 mg (100.9%) | | |
| 3 | 201.9 mg (100.9%) | | 85.7% |
| 12 | 213.4 mg (106.7%) | | 86.1% |

TABLE 9
The potency, content uniformity, and
dissolution data for the 80 kg batch.

| | Chondroitin Sulfate | | |
|---|---|---|---|
| | Potency | | Dissolution |
| Timepoint | (% of theoretical) | Content | Q = 75% |
| (months) | Specification: 85-115% | Uniformity | (at 60 min) |
| 0 | 206.5 mg (103.3%) | 0.33% RSD | 93.9% |
| 3 | 211.4 mg (105.7%) | | 93.4% |
| 6 | 208.4 mg (104.2%) | | 86.6% |
| 9 | 186.6 mg (93.3%) | | 91.8% |
| 12 | 196.0 mg (98.0%) | | 86.3% |
| 18 | 191.2 mg (95.6%) | | 83.4% |

TABLE 10
The average percent potency and relative standard
deviation of sodium chondroitin sulfate of 17 lots
taken from both the 20 kg and 80 kg batches.

| Timepoint (months) | Average Potency | Standard Deviation |
|---|---|---|
| 0 | 102.1 | 2.7 |
| 3 | 99.4 | 5.6 |
| 6 | 103.1 | 3.6 |
| 9 | 97.8 | 3.6 |
| 12 | 100.8 | 4.8 |
| 18 | 97.2 | 6.3 |
| 24 | 101.6 | 6.5 |

TABLE 11
The average dissolution values of chondroitin sulfate of
17 lots taken from both the 20 kg and 80 kg batches.

| | Average Dissolution | |
|---|---|---|
| Timepoint (months) | (Q = 75% at 60 min) | Standard Deviation |
| 0 | 93.3 | 22.2 |
| 3 | 92.7 | 30.4 |
| 6 | 88.8 | 27.8 |
| 9 | 91.1 | 34.0 |
| 12 | 92.9 | 21.5 |
| 18 | 88.2 | 27.6 |
| 24 | 95.4 | 28.1 |

The invention envisions a method of treating a connective tissue disease, injury, or condition such as osteoarthritis by administering orally to a human or other mammal any one of the compositions described herein. The composition can be administered in tablet or capsule

The invention provides a stable formulation and the compositions meet established specifications in terms of potency, content uniformity, and dissolution at time of manufacture (demonstrating manufacturability of formulations) and established specifications for potency and dissolution for at least 24 months after manufacture. Therefore, the invention advantageously provides the required characteristics of stability while being useful in the treatment of connective tissue disease and, more particularly, in osteoarthritis.

It is to be understood that the invention has been described with respect to certain specific embodiments thereof for purposes of illustration and not limitation. The present invention envisions that modifications, changes, and the like can be made therein such as colorants, pigments, disintegrating agents, binders, and other functional excipients without departing from the spirit and scope of the invention as set forth in the following claims.

1. A therapeutic composition comprising, in weight percent of the composition, an aminosugar from about 10 to about 70 percent, a glycosaminoglycan from about 10 to about 70 percent, a diluent from about 5 to about 80 percent, a glidant from about 0.02 to about 2 percent, and a lubricant from about 0.25 to about 5 percent.

2. The composition of claim 1 wherein the amount of the aminosugar is about 50 percent by weight.

3. The composition of claim 1 wherein the aminosugar is selected from the group consisting of glucosamine, glucosamine salts, and mixtures thereof.

4. The composition of claim 3 wherein the aminosugar is glucosamine hydrochloride.

5. The composition of claim 1 wherein the amount of the glycosaminoglycan is about 43 percent by weight.

6. The composition of claim 1 wherein the glycosaminoglycan is selected from a group consisting of chondroitin, chondroitin salts, and mixtures thereof.

7. The composition of claim 6 wherein the glycosaminoglycan is sodium chondroitin sulfate.

8. The composition of claim 1 wherein the amount of the diluent is about 6 percent by weight.

9. The composition of claim 1 wherein the diluent is selected from the group consisting of microcrystalline cellulose, starch, calcium phosphate, calcium carbonate, detroxe, detrines, dextrates, lactose, mannitol, sucrose, silicified microcrystalline cellulose, cellulose, and mixtures thereof.

10. The composition of claim 1 wherein the amount of the glidant is about 0.5 percent by weight.

11. The composition of claim 1 wherein the glidant is selected from the group consisting of fumed silica, talc, cornstarch, magnesium, silicate, magnesium trisilicate, and mixtures thereof.

12. The composition of claim 1 wherein the lubricant is about 0.5 percent by weight.

13. The composition of claim 1 wherein the lubricant is selected from the group consisting of magnesium stearate, stearic acid, hydrogenated castor oil, talc, glycerin monostearate, magnesium lauryl sulfate, zinc stearate, polyethylene glycol, and mixtures thereof.

14. The composition of claim 1 is stable for at least 24 months after the date of manufacture.

15. A therapeutic composition comprising, in weight percent of the composition, an aminosugar from about 20 to about 95 percent, a diluent from about 5 to about 95 percent, a glidant from about 0.02 to about 2 percent, and a lubricant from about 0.25 to about 5 percent.

16. The composition of claim 15 wherein the amount of the aminosugar is about 55 percent by weight.

17. The composition of claim 15 wherein the aminosugar is selected from the group consisting of glucosamine, glucosamine salts, and mixtures thereof.

18. The composition of claim 17 wherein the aminosugar is glucosamine hydrochloride.

19. The composition of claim 15 wherein the amount of the diluent is about 44 percent by weight.

20. The composition of claim 15 wherein the diluent is selected from the group consisting of microcrystalline cellulose, starch, calcium phosphate, calcium carbonate, detroxe, detrines, dextrates, lactose, mannitol, sucrose, silicified microcrystalline cellulose, cellulose, and mixtures thereof.

21. The composition of claim 15 wherein the amount of the glidant is about 0.5 percent by weight.

22. The composition of claim 15 wherein the glidant is selected from the group consisting of fumed silica, talc, cornstarch, magnesium, silicate, magnesium trisilicate, and mixtures thereof.

23. The composition of claim 15 wherein the lubricant is about 0.5 percent by weight.

24. The composition of claim 15 wherein the lubricant is selected from the group consisting of magnesium stearate, stearic acid, hydrogenated castor oil, talc, glycerin monostearate, magnesium lauryl sulfate, zinc stearate, polyethylene glycol, and mixtures thereof.

25. The composition of claim 15 is stable for at least 24 months after the date of manufacture.

26. A therapeutic composition comprising, in weight percent of the composition, a glycosaminoglycan from about 20 to about 95 percent, a first diluent from about 5 to about 95 percent, a second diluent from about 5 to about 95 percent, a glidant from about 0.02 to about 2 percent, and a lubricant from about 0.25 to about 5 percent.

27. The composition of claim 26 wherein the amount of the glycosaminoglycan is about 43 percent by weight.

28. The composition of claim 26 wherein the glycosaminoglycan is selected from a group consisting of chondroitin, chondroitin salts, and mixtures thereof.

29. The composition of claim 28 wherein the glycosaminoglycan is sodium chondroitin sulfate.

30. The composition of claim 26 wherein the amount of the first diluent is about 16 percent by weight.

31. The composition of claim 26 wherein the first diluent is selected from the group consisting of dibasic calcium phosphate, starch, microcrystalline cellulose, calcium carbonate, dextroxe, dextrines, dextrates, lactose, mannitol, sucrose, silicified microcrystalline cellulose, cellulose, and mixtures thereof.

32. The composition of claim 26 wherein the amount of the second diluent is about 40 percent by weight.

33. The composition of claim 26 wherein the second diluent is selected from the group consisting of microcrystalline cellulose, starch, calcium phosphate, calcium carbonate, detroxe, detrines, dextrates, lactose, mannitol, sucrose, silicified microcrystalline cellulose, cellulose, and mixtures thereof.

34. The composition of claim 26 wherein the amount of the glidant is about 0.5 percent by weight.

35. The composition of claim 26 wherein the glidant is selected from the group consisting of fumed silica, talc, cornstarch, magnesium, silicate, magnesium trisilicate, and mixtures thereof.

36. The composition of claim 26 wherein the lubricant is about 0.5 percent by weight

37. The composition of claim 26 wherein the lubricant is selected from the group consisting of magnesium stearate, stearic acid, hydrogenated castor oil, talc, glycerin monostearate, magnesium lauryl sulfate, zinc stearate, polyethylene glycol, and mixtures thereof.

38. The composition of claim 26 is stable for at least 24 months after the date of manufacture.

39. A therapeutic composition comprising, in weight percent of the composition, glucosamine hydrochloride in an amount of about 50 percent, sodium chondroitin sulfate in an amount of about 43 percent, microcrystalline cellulose in an amount of about 6 percent, fumed silica in an amount of about 0.5 percent, and magnesium stearate in an amount of about 0.5 percent.

40. A therapeutic composition comprising, in weight percent of the composition, glucosamine hydrochloride in an amount of about 55 percent, microcrystalline cellulose in an amount of about 44 percent, fumed silica in an amount of about 0.5 percent, and magnesium stearate in an amount of about 0.5 percent.

41. A therapeutic composition comprising, in weight percent of the composition, sodium chondroitin sulfate in an amount of about 43 percent, dibasic calcium phosphate in an amount of about 16 percent, microcrystalline cellulose in an amount of about 40 percent, fumed silica in an amount of about 0.5 percent, and magnesium stearate in an amount of about 0.5 percent.

42. A method of treating a connective tissue disease, injury, or condition comprising administering to a mammal the composition of claim 1.

43. The method of claim 42 wherein the composition is in tablet form.

44. The method of claim 42 wherein the composition is in capsule form.

45. The method of claim 42 wherein the connective tissue disease is osteoarthritis.
